(19) United States
(12) Patent Application Publication
(10) Pub. No.: US 2005/0196437 A1
(43) Pub. Date: Sep. 8, 2005

(54) Hard capsules

(76) Inventors: Christina A. Bednarz, Somerville, NJ (US); Bodan Ma, Racine, WI (US); James J. Kasica, Whitehouse Station, NJ (US); Monika K. Okoniewska, Princeton, NJ (US); Zhixin Li, Bridgewater, NJ (US)

Correspondence Address: Karen G. Kaiser;NATIONAL STARCH AND CHEMICAL COMPANY, 10 Finderne Avenue, Bridgewater, NJ, 08807-0500, US

(21) Appl. No.: 10/791,540

(22) Filed: Mar. 2, 2004

(51) Int. Cl. A61K 31/717; A61K 9/28; A61K 9/68

(52) U.S. Cl. 424/451; 514/60

(57) ABSTRACT

The present invention pertains to a blend of a physically induced starch hydrolysate, a plasticizer, and a gelling agent. Such blend produces an excellent film with low brittleness. Further, essentially gelatin-free hard capsules may be made with such blends.

### BACKGROUND OF THE INVENTION

The present invention relates to a method of producing a film-forming alternative to gelatin, and its use in making hard, essentially gelatin-free films and capsules which have similar textural and functional properties compared to hard capsules made using gelatin.

Gelatin is used in various pharmaceutical applications including soft gelatin capsules and hard gelatin capsule shells as well as many different food applications. Hard capsules are used to encapsulate a dry substance such as a powder or microcapsules, for delivery of such substance, for example of a nutritional or pharmaceutical active agent. Hard capsules have many advantages over other dosage forms, permitting accurate delivery of a unit dose in an easy-to-swallow, transportable, essentially tasteless form.

However, gelatin has many drawbacks, including the cost and continuity of a safe supply. Bovine sources are also undesirable to certain individuals, such as vegetarians and those wishing to maintain Kosher or Halal standards. Further, gelatin is prone to crosslinking, caused by aging or due to reaction with compounds such as aldehydes, which reduces its solubility in gastric juices.

Gelatin provides a good capsule as the film is strong and elastic enough to survive the manipulation of filling and transportation, yet dissolves in gastric juices. With the growing concern of Bovine Spongiform Encephilitis (BSE) disease in products derived from cows, many attempts have been made to replace gelatin, such as that in hard capsules. However, these approaches have typically failed in that the resultant products had unacceptably different textural and/or functional properties.

Surprisingly, it has now been found that the blend of a physically induced starch hydrolysate, a plasticizer, and a gelling agent gives an excellent film with a high modulus and excellent toughness. Further, essentially gelatin-free hard capsules may be made with such blends.

### SUMMARY OF THE INVENTION

The present invention pertains to a blend of a physically induced starch hydrolysate, a plasticizer, and a gelling agent. Such blend produces an excellent film with low brittleness. Further, essentially gelatin-free hard capsules may be made with such blends.

### DETAILED DESCRIPTION OF THE INVENTION

The present invention pertains to a blend of a physically induced starch hydrolysate, a plasticizer, and a gelling agent. Such blend produces an excellent film with low brittleness. Further, essentially gelatin-free hard capsules may be made with such blends.

The blend comprises at least one starch. Starch, as used herein, is intended to include all starches derived from any native source, any of which may be suitable for use herein. A native starch as used herein, is one as it is found in nature. Also suitable are starches derived from a plant obtained by standard breeding techniques including crossbreeding, translocation, inversion, transformation or any other method of gene or chromosome engineering to include variations thereof. In addition, starch derived from a plant grown from artificial mutations and variations of the above generic composition, which may be produced by known standard methods of mutation breeding, are also suitable herein.

Typical sources for the starches are cereals, tubers, roots, legumes and fruits. The native source can be any variety of corn (maize), pea, potato, sweet potato, banana, barley, wheat, rice, oat, sago, amaranth, tapioca, arrowroot, canna, sorghum, and waxy and waxy varieties thereof. As used herein, “waxy” is intended to include a starch containing no more than about 10%, particularly no more than about 5%, more particularly no more than about 3%, and most particularly no more than about 1% amylose by weight. Waxy starches are particularly suitable for the present invention.

The starches must be at least partially pregelatinized using techniques known in the art and disclosed for example in U.S. Pat. Nos. 4,465,702, 5,037,929, 5,131,953, and 5,149,799. Also see, Chapter XXII—“Production and Use of Pregelatinized Starch”, Starch: Chemistry and Technology, Vol. III—Industrial Aspects, R. L. Whistler and E. F. Paschall, Editors, Academic Press, New York 1967.

Any starch having suitable properties for use herein may be purified by any method known in the art to remove starch off flavors and colors that are native to the polysaccharide or created during processing. Suitable purification processes for treating starches are disclosed in the family of patents represented by EP 554 818 (Kasica, et al.). Alkali washing techniques, for starches intended for use in either granular or pregelatinized form, are also useful and described in the family of patents represented by U.S. Pat. No. 4,477,480 (Seidel) and U.S. Pat. No. 5,187,272 (Bertalan et al.). Purification methods to remove other impurities or undesirable components, such as removal of protein by proteases, may also be used as desired.

The starch is physically induced to form a starch hydrolysate using methods known in the art. Physical treatment includes any method to mechanically alter the starch, such as by shearing, and, as used herein, is intended to include conversion. Methods of physical treatment known in the art include, without limitation, ball-milling, homogenization, high shear blending, high shear cooking such as jet-cooking or in a homogenizer, drum drying, chilsonation, roll-milling, and extrusion. In one embodiment, extrusion is the method of physically inducing the starch. The starches are not chemically modified.

In one embodiment, the starch is extruded, then optionally dried, ground and fractionated. In another embodiment, the starch is jet-cooked, such as with excess steam, and the starch is then recovered by any suitable method to dry the dispersed starch, including without limitation drum-drying and spray-drying.

Extrusion may be conducted using any suitable equipment and process parameters known in the art. Since a large number of combinations of process parameters exist, i.e. product moisture, screw design and speed, feed rate, barrel temperature, die design, formula and length/diameter (L/d) ratios, Specific Mechanical Energy (SME) and Product Temperature (PT) have been used in the art to describe the process parameter window of the extrusion. In one embodiment, an SME of 50 to 500 Wh/kg and a PT of 50 to 220° C. are used. In another embodiment, an SME of 150 to 400 Wh/kg and a PT of 120 to 220° C. are used. In yet another embodiment, an SME of 200 to 350 Wh/kg and a PT of 160 to 190° C. are used. However, it is important to use an SME and PT combination, which will result in decreased molecular weight.

The starch is physically induced to a dextrose equivalence of no more than about 1. Dextrose equivalence (DE), as used herein, is intended to mean the reducing power of a starch hydrolyzate. Each starch molecule has one reducing end: therefore DE is inversely related to molecular weight. The DE of anhydrous D-glucose is defined as 100 and the DE of unhydrolyzed starch is virtually zero.

The resultant physically induced (treated) starches may be rendered pregelatinized by the physical treatment such that they are easily hydrated. If they are not, the starch must be cooked (gelatinized) using methods known in the art prior to film formation.

The physically induced starches may be characterized by good hydration properties, viscosity stability in solution at 70° C. as evidenced by dispersed solutions (no significant settling), and dried films that remain substantially clear to translucent upon formulation.

The starch may be used in any amount necessary to achieve the desired viscosity and film thickness. In one embodiment, the starch is used in an amount of about 70 to 99.8%, in another at about 85 to 95%, by weight of the composition on a dry basis.

The blend also contains a plasticizer which may be any plasticizer known in the art. The plasticizer is intended to include, without limitation, polyhydric alcohols such as glycerin (glycerol), sorbitol, maltitol, propylene glycol, and polyethylene glycol, saccharides such as fructose, sucrose and corn syrup, and polysaccharide. In one embodiment, the plasticizer includes glycerin and in another is glycerin. The plasticizer may be used in any amount necessary to achieve the desired plasticizing effect and reduce the brittleness of the film. In one embodiment, the plasticizer is present in an amount of about 0.01 to 15%, in another about 5 to 10%, by weight of the composition on a dry basis.

The blend also contains a gelling agent. Gelling agents, as used herein, are intended to include without limitation alginates, agar gum, guar gum, locust bean gum, carrageenan, tara gum, gum arabic, gum ghatti, Khaya grandifolia gum, tragacanth gum, karaya gum, pectin, arabian, xanthan, gellan, and other exocellular polysaccharides. In one embodiment, gellan gum is used as the gelling agent and in another low acyl gellan gum. The gelling agent may be used in any amount necessary to achieve the desired gel strengthening effect, both modulus and toughness. In one embodiment, the gelling agent is used in an amount of about 0.1 to 15%, in another at about 1 to 10%, by weight of the composition on a dry basis.

Gellan gum, as used herein, refers to the extracellular polysaccharide obtained by the aerobic fermentation of the microorganism Pseudomonas elodea in a suitable nutrient medium. Various forms of gellan gum have been described in the art and may be used in the present invention.

As used herein, low acyl content is intended to mean less than 25% acetyl and less than 15% glyceryl residual substituents per repeat unit. The low acyl gellan increases the rigidity in the end use application.

Synthetic polymers may be added to the formulation in an amount of about 0 to 10%, by weight of the composition on a dry basis. Suitable synthetic polymers include polyvinyl pyrrolidone (either as a separate ingredient or with coprocessing).

Other additives may optionally be included in the film as is common in the industry as long as they do not adversely affect the film, including without limitation colors, flavors, preservatives, opacifying agents, embrittlement inhibiting agents, disintegrants and buffers. In one embodiment, the blends are essentially gelatin-free, in another they contain less than about <0.1% gelatin.

If the dry blend contains a pregelatinized starch, the blend may be directly added to water to form a solid concentration suitable for the film or capsule process used. In the alternative, the blend may be cooked prior to formation of the film or hard capsule.

In one embodiment, the blend is added to deionized water. Adding salt may further improve the mechanical properties and moisture retention. In one embodiment, monovalent ions, such as sodium and potassium, are added in an amount of 10 to 100 millimolar in a solution containing about 20% starch. In another embodiment, divalent ions, such as calcium and magnesium, are added in an amount of 5-30 millimolar in a solution containing about 20% of starch. An excess of cations, especially divalent cations, may slow dissolution of the finished films.

The dispersion or solution may then be made either into a film or a hard capsule using methods known in the art. The solids concentration of such dispersion or solution in one embodiment is at 15% to 35% solids. The blend is useful for forming essentially gelatin-free hard capsules, for example by conventional dip molding processing. Other uses for the films include agricultural applications (including herbicides, pesticides, and the like), detergent and personal care applications.

The resultant films have a modulus of from about 0.5 to 4.0 gPa and a relative film strength of about 0.05 to 0.4 gPa, as tested using the burst test on the Texture Analyzer (TA-XT21).

The resultant capsules will be similar in look and feel to gelatin-containing hard capsules and may be filled using materials typically used in the art, including powders, microencapsulations, and other dry actives. Fill materials may include without limitation nutriceuticals and pharmaceuticals.

### EXAMPLES

The following examples are presented to further illustrate and explain the present invention and should not be taken as limiting in any regard. All percents used are on a dry weight basis. All water used is deionized.

The following tests are used throughout the examples: Compositions were prepared by dissolving dry components into solution. A total mass of 100 g of slurry was used. The mixture was held for up to 30 minutes at 100° C. The mixture was stirred using an overhead stirrer at a speed of 100 rpm. The viscosity is measured using the Brookfield Model DV II+ viscometer with the Model 74R Temperature Controller. All measurements were taken at 70° C.

Film samples were created from the formulation by drawing a Bird applicator or similar device with an opening of 50 mils over a 4-mil PET substrate. The films were dried overnight on the bench-top before being cut into strips and stored in a humidity cabinet for equilibration.

Film characterization was performed on the TA-XT2i Texture Analyzer (hereafter Texture Analyzer).

The following ingredients are used throughout the examples. The gellan gum used in these examples was Gelrite Gellan Gum from commercially available from Sigma-Aldrich Corp., St. Louis, Mo., USA. The glycerin used in these examples is Glycerin EP/USP, commercially available from EMD Chemicals, Gibbstown, N.J.

### Example 1

### Preparation of the Blend

A starch was prepared using a waxy corn base and subjecting it to extrusion at a feed rate of 10 kg/hr, temperature 123° C., and 45% torque high end, 50% torque low end. SME was 213.8 Whrs/kg. The screw speed was 250 rpm. The starch was recovered and mixed (17.31% starch) into solution along with 0.75% gellan gum; 2.00% glycerin; 79.94% added water.

### Example 2

### Variations of the Blend

a. A variation of Example 1 was prepared except that:
40.09% extruded starch;
0.76% gellan gum;
2.00% glycerin; and
57.15% added water were used.

b. The blend of Example 1 was prepared except that:
15.04% extruded starch;
0.76% gellan gum;
5.00% glycerin; and
79.20% added water were used.

c. The blend of Example 1 was prepared except that:
31.47% extruded starch;
0.91% gellan gum;
3.02% glycerin; and
64.60% added water were used.

d. The blend of Example 1 was prepared except that:
17% extruded starch;
0.2% gellan gum;
2.00% glycerin; and
80.80% added water were used.

e. The blend of Example 1 was prepared except that:
17% extruded starch;
0.75% gellan gum;
0% glycerin;
82.25% added water were used.

f. The blend of Example 1 was prepared except that:
17% extruded starch;
0.75% gellan gum;
4.00% glycerin; and
78.25% added water were used.

g. The blend of Example 1 was prepared except that:
15% extruded starch;
0.75% gellan gum;
2.00% glycerin; and
82.25% added water were used.

h. The blend in example 1 was prepared except that:
17.25% extruded starch;
0.75% gellan gum;
10.00% glycerin; and
72.00% added water was used.

i. The blend in example 1 was prepared except that:
17.31% extruded starch
0.75% agar agar (in place of gellan gum)
2.00% glycerin; and
79.94% added water were used.

j. The blend in example 1 was prepared except that:
17.31% extruded starch
0.75% agar agar (in place of gellan gum)
2.00% sorbitol (in place of glycerin); and
79.94% added water were used.

### Example 3

### Preparation of Capsule Shells

Blends of Example 1 were used to make capsule shells as follows.

The formulation slurry was prepared by mixing the ingredients, cooking in a steam bath for 30 minutes with agitation. The agitation was then removed and the formulation was cooked for an additional 30 minutes in the steam bath to remove bubbles. The formulation was then transferred to the dipping vessel at 70° C.

Stainless steel dipping pins were wiped with a release aid, such as vegetable oil. The pins were lowered slowly into the solution, and were slowly withdrawn, allowing excess solution to flow freely from the pins; and to prevent stringiness.

Once cooled and dried, the capsules were removed from the pins. The capsules were continued to be dried in a controlled humidity chamber until they reached a moisture content of around 10%.

Typically, the film thickness varied from 0.07 mm to 1.5 mm though this is not a necessary aspect of the invention.

### Example 4

### Properties of the Films

The films of Example 1 and 2 were tested for modulus, using a Texture Analyzer. A test method was created based upon the case studies TA Study I-95 Film Resilience, Springiness, & Burst Strength. The 5-kg load cell was installed inside the load cell arm.

The burst strength test was conducted by using the TA-108s Film/Gel Extensibility Fixture and the TA-8A ⅛″ (0.3175 cm) diameter stainless steel ball probe. The film was placed between the two washers of the Film/Gel Extensibility Fixture and was held in place by tightening the four screws. The sample film was cut into smaller pieces, and the tested area is circular with a diameter of 14.5 mm. This method was used because it provides more consistent results in comparison to the tensile test.

Results for Examples 1 and 2 are given below:

| | Max | | Elongation | Formulation |
|---|---|---|---|---|
| | Stress | Modulus | at Break | Viscosity |
| Example | gPa | GPa | % | cPs |
| 1 | 0.168 | 1.62 | 10.4 | 3300 |
| | 0.132 | 1.26 | 10.4 | 3900 |
| | 0.06 | 0.6 | 10.4 | 1700 |
| 2a | 0.054 | 1.08 | 4.9 | 31600 |
| 2b | 0.114 | 2.7 | 4.3 | 10000 |
| 2c | 0.072 | 1.86 | 3.8 | 11700 |
| 2d | 0.066 | 4.08 | 1.6 | 400 |
| 2e | 0.102 | 5.16 | 2.0 | 900 |
| 2f | 0.066 | 3.06 | 2.1 | 1100 |
| 2g | 0.084 | 4.14 | 2.1 | 600 |

In one suitable embodiment, as can be seen from the above chart, the viscosity is 900-2000 cps, the modulus is 1 to 2.5 gPa and the maximum elongation is about 10%.

In this measurement, the values for maximum stress were obtained by dividing the maximum force by the median cross section (perpendicular to the radial direction) of the film, and then normalized by a factor to the same form as the tensile test. The values for elongations were reduced using the stretched film radius divided by the radius of the original film.

### Example 5

### Jet Cooking and Spray Drying

Waxy corn starch was slurried at 25% anhydrous solids in deonized water. The slurry was jet-cooked using C1 jet-cooker available from NSC at 1034 kPa (150 psi) house steam pressure, 758 kPa (110 psi) inlet steam pressure. The slurry pump speed was set to 40%. The air-back pressure was set to 275.8 kPa (40 psi) and the steam flow was adjusted to 45% or 66% resulting in steam flow rates of 0.95 and 1.7 liters/min (0.25 and 0.45 gal/min). Temperature was controlled from 130 to 142° C. Dispersed starch was collected, and the solids were adjusted to 8 and 10% as is. The dispersed starch was then recovered by spray drying. Spray drying was performed on a Niro Spray Dryer with the two fluid nozzle. Drying parameters were set to an inlet temperature of 210° C. and a peristaltic pump speed of 95 rpm. The outlet temperature fluctuated between 110° C. to 115° C. Each slurry was introduced directly into the nozzle through the peristaltic pump.

| Waxy | | | Final | Spray | Final |
|---|---|---|---|---|---|
| Corn | Steam | Temp | Cook | Drying | Viscosity |
| Batch | Flow (%) | (° C.) | Solids (%) | Solids (%) | (cps) |
| A | 45 | 130 | 15 | 10 | 1900 |
| A | 66 | 130 | 18 | 10 | 1500 |
| B | 45 | 142 | 22 | 10 | 970 |
| B | 66 | 142 | 21 | 10 | 670 |
| C | 45 | 140 | 20 | 8 | 1060 |
| C | 66 | 140 | 19 | 8 | 860 |

Example 1 is repeated substituting each of the above starches for that of Example 1 and films are made.

1. A composition comprising:
(a) a physically induced starch hydrolysate;
(b) a plasticizer; and
(c) a gelling agent.

2. The composition of claim 1, wherein the starch is a waxy starch.

3. The composition of claim 1, wherein the starch is jet cooked.

4. The composition of claim 1, wherein the starch is extruded.

5. The composition of claim 4, wherein the extrusion is carried out using an SME of 50 to 500 Wh/kg and a PT of 50 to 220° C.

6. The composition of claim 4, wherein the extrusion is carried out using an SME of 150 to 400 Wh/kg and a PT of 120 to 220° C.

7. The composition of claim 4, wherein the extrusion is carried out using an SME of 200 to 350 Wh/kg and a PT of 160 to 190° C.

8. The composition of claim 1, wherein the dextrose equivalent of the starch is no more than about 1.

9. The composition of claim 1, wherein the starch is present in an amount of about 70 to 99.8% by weight of the composition on a dry basis.

10. The composition of claim 9, wherein the starch is present in an amount of about 85 to 95% by weight of the composition on a dry basis.

11. The composition of claim 1, wherein the plasticizer is glycerin.

12. The composition of claim 1, wherein the plasticizer is present in an amount of about 0.01 to 15% by weight of the composition on a dry basis.

13. The composition of claim 1, wherein the plasticizer is present in an amount of about 5 to 10% by weight of the composition on a dry basis.

14. The composition of claim 1, wherein the gelling agent is selected from the group consisting of gellan gum.

15. The composition of claim 14, wherein the gellan gum is a low acyl gellan gum.

16. The composition of claim 1, wherein the gelling agent is present in an amount of about 0.1 to 15% by weight of the composition on a dry basis.

17. The composition of claim 1, wherein the gelling agent is present in an amount of about 1 to 10% by weight of the composition on a dry basis.

18. A composition comprising:
(a) a waxy, physically induced starch hydrolysate with a dextrose equivalent of no more than about 1;
(b) glycerin; and
(c) gellan gum.

19. The composition of claim 18, wherein the starch is present in an amount of about 85 to 95%, the glycerin is present in an amount of about 0.01 to 15%, and the gellan gum is present in an amount of about 1 to 10%, by weight of the composition on a dry basis.

20. A film comprising:
(a) a waxy, physically induced starch hydrolysate with a dextrose equivalent of no more than about 1;
(b) glycerin; and
(c) gellan gum.

21. The film of claim 20, wherein the starch is present in an amount of about 85 to 95%, the glycerin is present in an amount of about 0.01 to 15%, and the gellan gum is present in an amount of about 1 to 10%, by weight of the composition on a dry basis.

22. A hard capsule comprising:
(a) a waxy, physically induced starch hydrolysate with a dextrose equivalent of no more than about 1;
(b) glycerin; and
(c) gellan gum.

23. The capsule of claim 22, wherein the starch is present in an amount of about 85 to 95%, the glycerin is present in an amount of about 0.01 to 15%, and the gellan gum is present in an amount of about 1 to 10%, by weight of the composition on a dry basis.

24. The capsule of claim 22, wherein the capsule is essentially gelatin-free.
